(19) United States
(12) Patent Application Publication
(10) Pub. No.: US 2007/0027459 A1
(43) Pub. Date: Feb. 1, 2007

(54) Method and system for configuring and data populating a surgical device

(76) Inventors: Christopher Horvath, Irvine, CA (US); Steven T. Charles, Memphis, TN (US); Edward G. Malk, Gilbert, AZ (US)

Correspondence Address: ALCON, IP LEGAL, TB4-8, 6201 SOUTH FREEWAY, FORT WORTH, TX, 76134, US

(21) Appl. No.: 11/492,616

(22) Filed: Jul. 25, 2006

Related U.S. Application Data
(60) Provisional application No. 60/703,724, filed on Jul. 29, 2005.

(51) Int. Cl. A61B 17/04; A61M 5/178

(52) U.S. Cl. 606/147; 604/164.08

(57) ABSTRACT

A system and method for configuring a surgical device are disclosed, one embodiment of the system comprising; an interface operable to receive data; a portable component, comprising an identifier operable to store and transmit a set of data; a memory, the memory storing criteria; and a controller, wherein the controller, the memory and the interface are included in the surgical device, and wherein the controller is operable to configure the surgical device based on the set of data transmitted from the identifier and received at the interface. The identifier can be an RFID tag and the interface can be an RFID interface comprising an RFID reader operable to transmit a radio frequency (RF) instruction to activate the RFID tag of the portable component, and a microcontroller operable to cause the RFID reader to transmit the RF instruction. The portable component can be an identification tag that can be attached to the user. The criteria can be user authorization data that is stored in the memory and the controller is operable to determine whether a user associated with the portable component is authorized to use the surgical device based on whether the set of data transmitted from the identifier and received at the interface satisfies the criteria stored in the memory. The criteria can also be an algorithm that the controller applies to the set of data received from the identifier to determine whether the user is authorized to use the surgical device. The controller can cause the surgical device to be disabled if it is determined that the user is not authorized to use the surgical device (e.g., the surgeon, technician or patient is at the wrong surgical device).

### CROSS-REFERENCE TO RELATED APPLICATIONS

This application claims priority under 35 U.S.C. §119 to U.S. Provisional Patent Application No. 60/703,724, filed Jul. 29, 2005, the entire contents of which are incorporated herein by reference.

### TECHNICAL FIELD OF THE INVENTION

The present invention relates to surgical procedures performed using surgical devices, and, more particularly, to methods and systems for configuring and data-populating a surgical device. Even more particularly, the present invention relates to methods and systems using electronic identifiers to transmit and receive data for configuring and data-populating a surgical device.

### BACKGROUND OF THE INVENTION

Ensuring that a patient receives the intended treatment for a medical condition is an ultimate goal of the medical profession. In particular, ensuring that a patient does not undergo an erroneous surgical procedure, or a correct surgical procedure on the wrong body part, is of paramount importance for the health and safety of the patient. It is well known that such errors occur. These situations are not only unfortunate, but can have disastrous and even deadly consequences for the patient. Further, the liability incurred by the surgical provider can be great. Although the exact number of such erroneous procedures is unknown, it is an ultimate goal to reduce and preferably eliminate the opportunity for error when it comes to matching a patient with an intended surgical procedure.

The number and complexity of surgical procedures is increasing every day, with a resultant increase in the likelihood that an error, such as described above, will occur. Currently, most such errors are avoided by conscious and rigid requirements on surgical personnel to cross-check the identity of a patient and the important data associated with the patient against the intended surgical procedure and surgical device. However, even the most rigid requirements on personnel cannot eliminate the likelihood of human error, especially when dealing with a large number of patients in a rapidly moving and hurried clinical environment. Typically, cross-checking a patient to an intended surgical procedure and surgical device consists of checking a patient's identification bracelet to a chart. Under such circumstances, the possibility exists that a patient may be assigned a wrongly labeled bracelet, or that the patient is correctly matched to a procedure, but the procedure is performed on the wrong body part or using the wrong parameters on a given surgical device. Positively identifying a patient and matching the patient to the correct surgical procedure using the correct parameters for an intended surgical device, on the correct body part, is thus becoming increasingly important. Current methods for configuring and data-populating a surgical device to ensure a positive patient-surgery match are not sufficiently effective and reliable in today's surgical climate.

One method for identifying and confirming a patient-surgery match is disclosed in U.S. patent application Ser. No. 10/737,609, the contents of which are hereby incorporated by reference in their entirety. Biometric identification systems including inkless fingerprint systems, called “live-scan units”, retinal scanners, hand geometry measuring devices, voice recognition, handwriting recognition and facial recognition systems that use either visual or infrared cameras are reliable and, because of this, are typically used for access control and for tracking or identifying persons following some event. These types of systems, however, while improving security of the patient-to-surgical procedure match, do not alone provide a means for configuring or data-populating a surgical device prior to a surgical procedure. Another goal of the medical profession is to improve the efficiency and reduce the time (and consequently, the cost) of a surgical procedure. The ability to automate aspects of the configuration and data entry of a surgical device can greatly further this goal.

Beyond systems and methods for improving the safety of a surgical procedure by ensuring correct patient/surgical procedure/surgical device matching, improved systems and methods are thus also needed for streamlining the configuration and data-population of a surgical device in preparation for a surgical procedure. Surgical devices require certain inputs before surgery can be performed. For example, patient data (e.g., name, age, sex, etc.), surgical data (e.g. type of surgery, body part, etc.), device settings (e.g., power, duration, etc.), confirmation of the surgeons preferred settings, service personnel inputs (e.g., calibration and maintenance records, software updates) and other such inputs have to be confirmed as up-to-date. Similarly, surgical devices also provide outputs, such as a patient record of the performed procedure(s) including the surgeon's name and device, diagnostic, maintenance and calibration settings, etc., that are stored in the device and provided as outputs to, for example, establish a separate record or to provide a read-out to a user.

Surgical device inputs and outputs are typically performed via a keyboard and a mouse and/or file transports through a USB port, ethernet, a printer, CD, floppy, etc. In other words, input/outputs are typically manually captured via an interface and originate as recorded entries by a surgeon or his assistant during a pre-exam operative procedure and post-operative follow-up examinations and, in the case of service entries, recorded entries by a service technician. This type of information is typically maintained in hard-copy patient records and service records. While these methods are reliable and necessary (e.g., a paper record is desirable as a backup), they have the disadvantage of requiring additional manual steps before, during, and after a surgical procedure and/or during a service visit, thus requiring additional time and resources to perform. One result is the possibility for error both in reading (identifying) the entries and in inputting the information into a surgical device, as well as the concurrent delay in performing a surgical procedure because of the necessity of manually entering the data. The flow of a surgical procedure is thus disrupted and the efficiency and time-management of the surgeon is reduced.

There have been attempts to address some of these concerns, but only with specific types of medical equipment and with limited effectiveness. One approach has been to utilize radio frequency identification (“RFID”) systems. RFID systems are well known and use electronic tags or transponders for storing data. Some RFID systems use passive tags that are activated when they are brought into proximity to a transmitted radio signal, whereas other RFID systems use active tags that include a power source to operate independently.

RFID tags (devices) have been used with specific types of medical equipment but, to the applicant's knowledge, RFID technology has yet to be effectively applied to ophthalmic surgery systems and components or to other surgical systems and components. For example, one known system uses RFID devices in connection with disposal optical fiber components of a medical laser system in which fiber optic strands are inserted into the body. The strands are exposed to body fluids and must be disposed of after every use or thoroughly disinfected. Other known systems use RFID devices with catheters that are inserted into the vascular system and directed into the heart. These known systems, however, use RFID devices for particular surgical devices and do not provide for identifying individual patients, surgeons, or service personnel to a surgical device or for configuring and data-populating a surgical device.

Another known RFID system is used to track surgical implements. A sensor system records the time each surgical implement is checked out/used. When the surgical implement has been used, it is placed on or near the sensor and check-in information is recorded. This system is used to track surgical implements during a procedure and to insure that no medical implements are inadvertently left behind inside a patient.

Thus, known RFID systems and techniques do not provide for identifying patients or doctors or data-populating surgical equipment. Further, the use of RFID tags with other medical devices is typically limited to basic identification functions and enabling or disabling equipment. Thus, known systems do not provide other, more useful, data concerning a surgical device and its functionality or a patient and his/her required parameters, a surgeon's preferred equipment settings, and/or a service technician's maintenance information, or for synchronization of such data with a surgical device. For example, service information may include calibration data and data related to the history of the device. Accordingly, the manner in which patients, surgeons, and service technicians are identified to a surgical device and share information for configuring and data-populating the surgical device can be improved. For example, data transmission devices, such as RFID devices, can be incorporated to perform identification, configuration, data-population and other functions that are not provided by known systems, and can be used to provide information relating to the surgical device to users such as a surgeon or service personnel.

Therefore, a need exists for a method and system for configuring and data-populating a surgical device that can reduce or eliminate the problems associated with previous methods for matching a patient and/or a surgeon to a surgical procedure in a surgical environment. Further, a need exists for a method and system that can provide the same type of configuration and data exchange functionality for a service technician to improve the synchronization and matching of data between a patient, a surgeon and/or a service technician to a surgical device and/or surgical procedure.

### BRIEF SUMMARY OF THE INVENTION

Embodiments of the method and system for configuring and data-populating a surgical device of the present invention substantially meet these needs and others. One embodiment of this invention is a system for configuring a surgical device, comprising; an interface operable to receive data; a portable component, comprising an identifier operable to store and transmit a set of data; a memory, the memory storing criteria; and a controller, wherein the controller, the memory and the interface are included in the surgical device, and wherein the controller is operable to configure the surgical device based on the set of data transmitted from the identifier and received at the interface. The identifier can be an RFID tag and can also be operable to receive data and the interface can be an RFID interface comprising an RFID reader operable to transmit a radio frequency (RF) instruction to activate the RFID tag of the portable component, and a microcontroller operable to cause the RFID reader to transmit the RF instruction. The portable component can be an identification tag that can be attached to the user.

The criteria can be user authorization data that is stored in the memory and the controller is operable to determine whether a user associated with the portable component is authorized to use the surgical device based on whether the set of data transmitted from the identifier and received at the interface satisfies the criteria stored in the memory. For example, the set of data received from the identifier is compared to the user authorization data at the controller to determine whether the user is authorized to use the surgical device. The criteria can also be an algorithm that the controller applies to the set of data received from the identifier to determine whether the user is authorized to use the surgical device. The controller can cause the surgical device to be disabled if it is determined that the user is not authorized to use the surgical device (e.g., the surgeon, technician or patient is at the wrong surgical device).

The interface can be operable to receive and transmit data and to program the identifier with a second set of data, which can be received and stored at the identifier. The two sets of data can be different. The set of data can be patient identification data, surgeon custom setting data, service data, maintenance data, surgical device calibration data, and surgical device configuration data, etc, and can be encrypted. Configuring the surgical device can comprise data-populating selected data fields based on the set of data, setting selected surgical device parameters to predetermined values in preparation for using the surgical device based on the set of data, and/or placing the surgical device in a service configuration in preparation for repair or maintenance.

Further embodiments of the present invention can include a method for configuring and data-populating a surgical device in accordance with the teachings of this invention. One embodiment can be a method for configuring a surgical device, comprising: providing a portable component, comprising an identifier operable to receive, store and transmit a set of data; establishing a criteria and storing the criteria in a memory of the surgical device; transmitting the set of data stored in the identifier from the identifier to an interface operable to receive data in the surgical device; determining at a controller in the surgical device whether a user associated with the portable component is authorized to use the surgical device based on whether the set of data transmitted from the identifier and received at the interface satisfies the criteria stored in the memory; and configuring the surgical device based on the set of data.

According to one embodiment of the present invention, a system for configuring and data-populating a surgical device includes a radio frequency identification (RFID) tag that can be attached to or carried by a user, a receiver and/or a transceiver, a memory for storing criteria and a controller, which are included in the surgical device. Data from the RFID tag is transmitted to the receiver in the ophthalmic surgical device. The controller receives the transmitted data and is operable to configure the surgical device in accordance with the data and to populate various data fields with portions of the transmitted data. The transmitted data can be encrypted or unencrypted and further can be matched to data stored in the surgical device memory to effectuate one or more process, security check or recognition steps. The various embodiments of the method and system of the present invention can be used together with the system for biometric surgical confirmation disclosed in U.S. patent application Ser. No. 10/737,609, the contents of which are hereby incorporated by referenced in their entirety, too provide further security in the patient/surgical procedure/surgical device match.

Yet a further alternative embodiment is a method of configuring and data-populating a surgical device that includes establishing criteria and storing criteria in a memory of the ophthalmic surgical device and transmitting data from an identifier in a tag or other wearable component to a receiver in the ophthalmic surgical device. Data received from the tag is processed to configure and/or data-populate the ophthalmic surgical device.

In various embodiments, the criteria or information can be authorized data, e.g. patient/surgeon/technician specific data, that is stored in the memory. The data that is received from the identifier (tag) is compared to the authorized data to determine whether, for example the patient is the correct patient, whether the surgeon and patient match up, whether the surgeon and treatment or patient and treatment match up, or whether a service technician is authorized to work on a particular machine, etc. For example, the surgical device can be enabled if the received data matches authorized data criteria. Further, the criteria can be an algorithm, a formula or other predefined criteria (generally “algorithm”). The algorithm can be applied to the data that is received from the tag identifier to determine whether the received data solves or satisfies the algorithm or formula and hence whether the surgeon, service technician or patient are properly matched to a given surgical device.

In various embodiments, different surgical devices and components can be utilized. For example, the ophthalmic surgical device can be a laser or laser console, or a vitro-retinal surgical device. Further, the receiver can be an RFID reader, the identifier tag can be an RFID tag and the data in the RFID tag or identifier can be encrypted or unencrypted.

System embodiments can be implemented to configure a surgical device for surgery and/or data-populate various data fields within the surgical device. If the data received from the identifier tag satisfies the criteria, then the device can be enabled, configured and data-populated. Safety precautions can be implemented in the embodiments of the present invention, such as generating a message at the surgical device to a surgeon or service technician that, for example, this is not the correct patient, the patient and intended treatment do not match, or that a surgical device requires a certain upgrade or maintenance be performed. Further, data can be written back to the identifier tag attached to the patient, surgeon, or service technician. The data may include the date of usage, patient information, procedure type, length of procedure, parameters of the machine, maintenance status, etc.

The various embodiments of an identifier tag can also include calibration data that indicate how the surgical device should be configured to work for a particular surgery, with a particular patient for a particular surgeon, etc. Identification, data, and/or calibration data can be used for various purposes. For example, a user interface that is presented on a display screen can be generated based on the particular surgical procedure that will be preformed. Data can also be used to enable operating parameters that are compatible with the intended procedure and the intended patient and to disable operating parameters that are incompatible with the intended procedure or that the surgeon does not desire to use. The data can be used to implement safety procedures, for example limiting the value of power, exposure range, and other operating parameters and checking whether a safety component, such as a filter that is associated with the identified procedure, is present.

Embodiments of the method and system of this invention can be implemented within any surgical environment, and, in particular, can be implemented within an ophthalmic surgery environment, such as for refractive surgery, cataract surgery and/or vitro-retinal surgery. For example, the embodiments of this invention can be implemented within the LADARVision® System, the ACCURUS® Surgical System, and/or the INFINITI® Vision System manufactured and sold by Alcon Laboratories, Inc, of Fort Worth, Tex. Other uses for the method and/or system for configuring and data-populating a surgical device of this invention will be known to those having skill in the art and are contemplated to be within the spirit and scope of the present invention.

### BRIEF DESCRIPTION OF THE SEVERAL VIEWS OF THE DRAWINGS

A more complete understanding of the present invention and the advantages thereof may be acquired by referring to the following description, taken in conjunction with the accompanying drawings in which like reference numbers indicate like features and wherein:

FIG. 1 is a simplified block diagram illustrating one embodiment of the system of the present invention;

FIG. 2, is a simplified block diagram of one embodiment of the present invention showing a system 10 in more detail;

FIG. 3 is a simplified block diagram generally illustrating one exemplary 1 RFID system 200 that can be used with the embodiments of the present invention;

FIG. 4 is a simplified block diagram illustrating in more detail an embodiment of this invention in which the RFID readers and tags are configured for two-way read/write applications;

FIG. 5 is a simplified block diagram of a surgical system 300 comprising one or more input devices 314 and 322 in accordance with the teachings of this invention; and

FIG. 6 is a conceptual drawing illustrating one potential implementation of an indicator for the location of a reader 220.

### DETAILED DESCRIPTION OF THE INVENTION

Preferred embodiments of the present invention are illustrated in the FIGUREs, like numerals being used to refer to like and corresponding parts of the various drawings.

The various embodiments of the present invention provide a system and method for configuring and data-populating a surgical system or device, such as a refractive laser eye surgery system, a vitro-retinal system, a cataract phacoemulsification system, or any other such surgical system. Embodiments of the method and system of this invention can comprise for example, a surgical system, such as known to those having skill in the art, coupled with a user interface for inputting and storing indicia of a patient and associating the patient's indicia with a desired surgical procedure. The surgical system or device can be a single piece, or a multiple piece system wherein the patient information can be acquired as a first step of a surgical procedure at a first unit of the surgical system and wherein the surgery itself can be performed at a second unit of the surgical system. The first and second units can be a single combined unit or separate units. The embodiments of the system of this invention can further comprise a programming interface at an input device for transferring and storing information onto a portable storage device. The programming interface can be a wireless interface or a wired interface for programming, for example, a portable identifier/tag. The tag is operable to wirelessly couple to the surgical device for transferring and receiving information to and from the surgical device. The surgical device can also comprise an input/output interface for interfacing with the portable unit (tag).

The portable tag can comprise, for example, an identification tag that can include a number, a patient photograph or some other means of identifying the tag as associated with a person, such as a particular patient, surgeon or service technician. The tag itself will also include, in a preferred embodiment, an RFID tag for storing, transmitting and receiving information between the tag and the surgical device and/or the input device. In a preferred embodiment, the interface between the surgical device and the portable tag is a wireless interface. The tag can be any portable component, including, for example, a keychain, a doll, a bracelet, etc., that can be attached to a user and that can incorporate the functionality disclosed herein. Embodiments of this invention can be used, without limitation, for patient data, surgeon's data, device configuration and maintenance data, and/or a service technician's custom data.

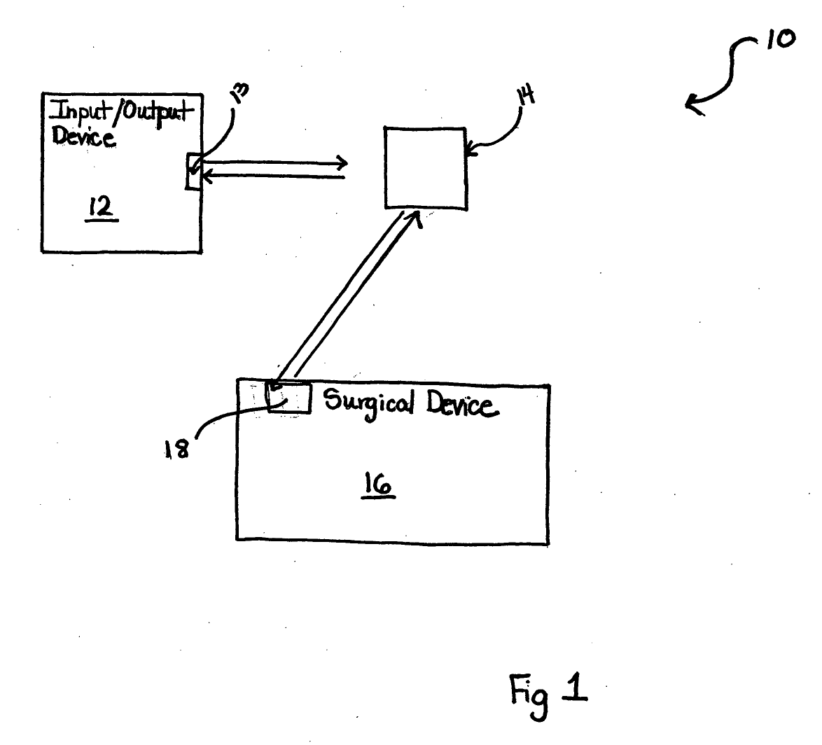

FIG. 1 is a simplified block diagram illustrating one embodiment of the present invention. System 10 comprises an input device 12, which can be either a stand-alone device or can be integrated into a surgical device or surgical console. Input device 12 includes an interface 13 for communicating (e.g., one-way or two-way) with portable tag 14. Interface 13 can comprise a wireless or a wired connection to program/populate the tag 14 with data such as patient identification data, surgeon preferences, surgical device parameters associated with a desired surgical procedure, and/or, in an embodiment including a service interface, a service technician's or surgical device's calibration data, maintenance data, upgrade data, etc. Tag 14 can be an RFID tag comprising, for example, an identification tag with an embedded RFID chip that can wirelessly couple to surgical device 16 and input device 12. Surgical device 16 can be, for example, a refractive laser system, a vitro-retinal system or a phacoemulsification system as known to those having skill in the art. Surgical device 16 includes interface 18 for receiving and/or transmitting information to and from tag 14.

In one embodiment, patient, surgeon, or service information can first be manually recorded (e.g., on paper or a separate device). For example, patient data can be taken during a diagnostic step, such as during an intake procedure, during a wavefront analysis for refractive surgery, or just as a stand-alone step for taking and associating patient data with a surgical procedure. The data is then input manually, via input device 12, and transferred/programmed onto tag 14 via interface 13. Tag 14 can be associated with a user (e.g., patient, surgeon, or service technician) via, for example, a photograph, number, or biometric data such as a fingerprint incorporated into tag 14. As an alternative to (or in addition to) manual input, previously recorded data can be electronically transferred to input device 12 in any manner known to those having skill in the art for later transfer to tag 14. Tag 14 can be attached to a patient (or other user) by, for example, a wrist strap, a neck strap, a clip or any other such means for attachment as known to those having skill in the art. Tag 14, now associated with the user, can be, for example, an ID tag that can be pre-made and can include a photo of the patient or other identifier (e.g., biometric information). Tag 14 remains with the patient as he/she progresses through the steps of a surgical procedure. If tag 14 is not present, the surgical device can provide an alert to indicate the missing condition.

Prior to the start of a surgical procedure, at surgical device 16, the patient's tag 14 is brought into close proximity to an interface 18, such as in the case of an RFID tag, and the information stored by tag 14 can be transferred to surgical device 16, where it is operable to cause surgical device 16 to be preconfigured and data fields data-populated based on the information stored on tag 14. In a similar fashion, surgical device 16 can be configured and/or data-populated by a tag 14 associated with a surgeon. Tag 14 can contain the surgeon's preferences for a particular device and/or a particular procedure and can also be preprogrammed via an input device 12 and interface 13. In such a case, tag 14 can store, for example, the surgeon's desired information for a particular patient, device, day or procedure or the surgeon's default configuration for a surgical device or devices.

Although the present invention is described here with reference to a single surgical device 16, it is contemplated to be within the scope of the invention that a single tag 14 can contain information for configuring and programming or data-populating multiple surgical devices 16. For example, if a surgical procedure requires more than one surgical device 16, the patient's tag 14 can store data for, and can be used to configure and data-populate, more than one surgical device 16. In a similar manner, a service technician's tag 14 can be configured with calibration data, maintenance data, update data, etc., for multiple surgical devices 16. Further, the information contained on tag 14 can be encrypted or non-encrypted with corresponding means for encrypting/un-encrypting the data at a surgical device 16 and at an input device 12. Encryption schemes, such as those disclosed in U.S. patent application Ser. No. 11/013,244, filed on Dec. 15, 2004, the contents of which are incorporated herein by reference in their entirety, can be used with the embodiments of the present invention.

The embodiments of the method and system for configuring and data-populating a surgical device of the present invention are discussed herein with regard to use in the general field of ophthalmic surgery. However, it is contemplated and will be realized by those skilled in the art that the scope of the present invention is not limited to ophthalmology, but may be applied generally to other areas of surgery where configuring and data-populating a surgical device prior to a surgical procedure may be required or desired. Further, embodiments of this invention can be used to configure non-surgical equipment in an analogous manner.

The embodiments of this invention provide for electronic identifiers integrated into a portable tag 14 that can be operably coupled to a surgical device 16 for configuring and/or data-populating the surgical device 16 prior to a surgical procedure. The electronic identifier is operable to transfer/receive/store data and to cause various functions to be performed, including; identifying a patient, a surgeon, or a service technician's information, and associating the patient, service technician or surgeon's information with a surgical device 16; selectively enabling and disabling equipment that is used with the surgical device 16; configuring parameters of the surgical device 16 for a surgical procedure or to a surgeon's preferences; and, transferring calibration data, maintenance data, upgrade or update information and service data between the tag 14 and the surgical device 16. Embodiments of this invention provide increased reliability and functionality over the prior art, thus improving the effectiveness and safety of a surgical procedure, such as an ophthalmic surgical procedure, by helping to ensure that patient/surgical procedure/surgical device match and that a surgeon's preferred configurations for a surgical device and/or surgical procedure are accurately and efficiently entered into the surgical device 16. These and other aspects of the embodiments are discussed in further detail below.

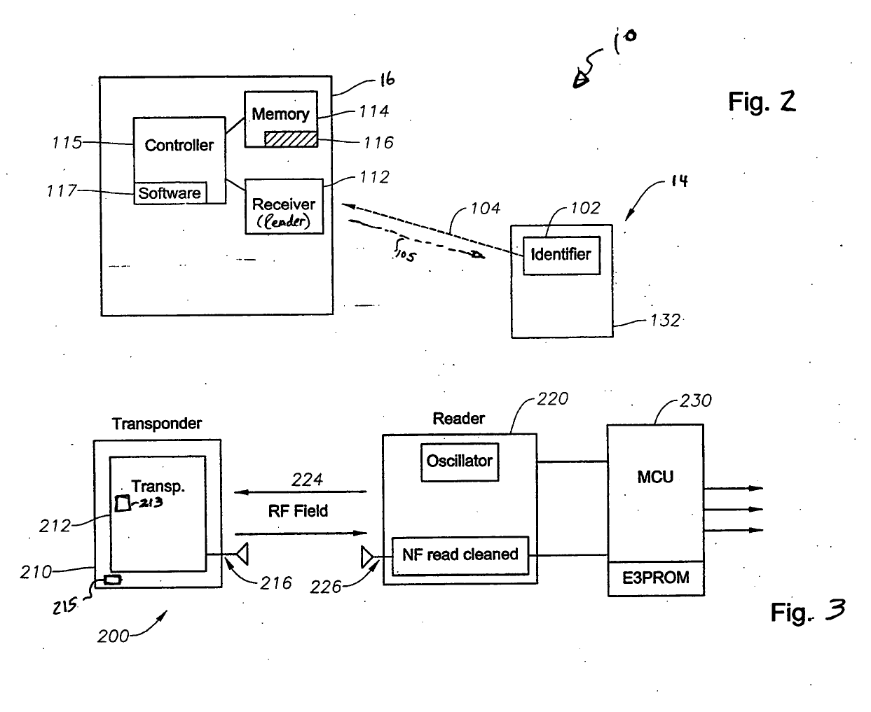

FIG. 2, is a simplified block diagram of one embodiment of the present invention showing a system 10 in more detail. System 10 includes a surgical device 16 and a portable tag 14 that is operable to be wirelessly coupled to surgical device 16. Tag 14 comprises an identifier 102. The identifier 102 transmits data 104 to (and can receive data 105 from) a receiver 112 of the surgical device 16. The data 104 can be used to configure surgical device 16 and/or to populate data fields associated with the patient, surgeon or service technician to which tag 14 has been assigned. Further, data 104 can be encrypted and can provide for a security function, such as determining whether the surgeon, technician or patient is authorized to use surgical device 16. Data 104 can comprise any of the various types of information described herein. In particular each tag 14 can be programmed with unique data 104.

Surgical device 110 includes a memory 114 that stores criteria 116, such as a corresponding set of authorized codes or data, or an algorithm, formula or other predefined criteria (generally “algorithm”). Memory 114 can be a memory element that is readily accessible or a memory element that is integrated within other system components, depending on security requirements. A controller 115, such as a processor or microcontroller (generally controller) is programmed with software or hardware 117 that processes the data 104 received from the identifier 102 and the criteria 116 stored in memory 114 to determine, for example whether the data 104 received from identifier 102 within tag 14 is associated with an authorized user, patient, doctor or service technician and to determine whether the surgical device 16 should be enabled or disabled. Further, controller 115 can be operable to generate control signals to surgical device 16 to configure surgical device 16 and data-populate selected data fields in accordance with data 104.

In one embodiment, the surgical device 110 is an ophthalmic laser console and identifier 102 is an RFID identifier. Tag 14 can be an identification tag associated with a patient, doctor or service technician and in each of these cases can contain appropriate data 104 identifying the patient and the surgical procedure, the surgeon and his or her preferred surgical parameters, or, in the case of a service technician, service data, surgical device information, updates etc. For example, surgical devices typically require the input of patient data such as the patient's name, date of birth, etc., patient parameter settings, the type of surgery, body location, device settings, etc., and to have the surgeon logged on to the device with his or her custom settings. Surgical devices can also provide several outputs, such as a patient record of the performed procedure after surgery, including the doctor's name and parameter settings and, in the case of a service call, diagnostic information, machine performance outputs, machine history, etc.

Embodiments of a patient tag 14 comprising a badge can be attached, for example, to a patient's arm or leg with a retractable cord that will allow the tag 14 to be pulled out a predetermined distance (e.g., five feet) sufficient to bring the tag 14 into proximity to surgical device 16 while remaining attached to the patient. The badge/tag 14 can store patient identification data as well as the patient's individual surgical machine settings. A corresponding surgeon's badge 14 could include data for logging the surgeon on to the surgical device 16, transferring his or her custom machine settings and configuring the surgical device 16 in accordance with those settings. Tag 14 can also be operable to receive data 105 from surgical device 16 and store the data 105, for example, in response to a surgeon's saving such data 105. A badge 14 can also provide a service technician the ability to transfer calibration and diagnostic data and software upgrades to and from a surgical device 16 and tag 14.

The embodiments of the method and system of this invention can be used within an entire product family, such as all surgical devices, from a manufacturer. Further, one embodiment contemplates a central input device 12 (e.g. in every hospital/office) where badges 14 can be programmed. The embodiments of the badge 14 of this invention can also be made disposable, requiring a new badge for each patient/surgical procedure. A person skilled in the art will appreciate that embodiments of this invention can be used with other surgical devices 16 and tags 14. As a further example, the surgical device 16 may be a vitrectomy console and the tag 14 can be a bracelet.

FIG. 3 is a simplified block diagram generally illustrating one exemplary RFID system 200 that can be used with the embodiments of the present invention. An RFID tag 210 (corresponding to identifier 102 of FIG. 2) typically includes an integrated circuit (IC) 215, such as an application specific integrated circuit (ASIC), that includes a memory for storing data. A transponder 212 is activated by radio frequency (RF) instruction or signal 224 from the reader 220. RF signal 224 is sent to (and then from) the reader antenna 226, for example, in response to a microcontroller 230 and received by an antenna 216 of the transponder 212 to wirelessly write data to or read data from the memory of the RFID tag 210. RFID tag 210 can be, for example, integrated within a tag 14 of FIGS. 1 and 2. Interfaces 13 and 18 of input device 12 and surgical device 16, respectively, can each comprise a Reader 220 and MCU 230.

For example, when RFID tag 210 is to be read, reader 220 sends out a, for example, 134.2 kHz power pulse to the antenna 226 lasting approximately 50 ms in one embodiment. The generated magnetic field is collected by the antenna 216 and the tag 210 that is tuned to the same frequency. This received AC energy is rectified and stored in a small capacitor 213 within the transponder 212. After completion of the power pulse, the transponder 212 transmits back its data using the energy stored in the capacitor 213 as a power source. In total, any number of bits can be transmitted over a specified period, for example, 20 ms. This data is received by the antenna 226 and decoded by the reader unit 220 and controller 230. The capacitor 213 is discharged after the data has been transmitted and the transponder 212 is reset and ready for the next read cycle. This is one embodiment, but any other RFID system as known to those having skill in the art can also be used with the embodiments of the present invention.

The RFID configuration described above with reference to FIG. 2 is passive, since the transponder 212 is powered from energy generated by the RF signal 224 from the reader 220 and stored in capacitor 213. Thus, a passive RFID identifier 102/210 is normally inactive and does not have an independent power source. The RFID system may also be active if a separate power source, such as a battery, is provided. Further details concerning the manner in which RFID systems operate are well known in the art and, therefore, are not discussed in this specification. For purposes of explanation, not limitation, this specification refers to RFID components that are used for transmitting data between a console surgical device 16 and a tag 14. However, a person skilled in the art will recognize that other transmitter, receiver and transceiver components can also be utilized.

In some embodiments of the present invention, it is contemplated that tag 14 is to be brought within a working range of approximately 1 inch from a reader 220 of a surgical device 16. This is to ensure that a positive confirmation is made between the tag 14 and reader 220 and that false confirmations or false transfers of data are not made between the reader 220 and other tags 14 that may be passing near the surgical device 16. For example, a patient and a surgeon may each have a tag 14 in accordance with this invention, but only one tag 14 at a time is read by bringing the tag 14 in close proximity to the reader 220. The working range between reader 220 and tag 14 can be adjusted, and longer ranges are contemplated to be within the scope of this invention, but is preferably in the range of about 1 to 2 inches.

In embodiments comprising RFID components to provide communications between a surgical device 16 and a tag 14, the identifier 102 in the tag 14 is an RFID tag or transponder 210 and the receiver 112 of the surgical device 16 is an RFID reader 220. The tag 14 for identifier 102 includes identification and, if applicable, other data relating to the user of the tag 14, as previously described. The controller 115 includes software and/or hardware 117 to implement the criteria 116 to determine whether data 104 sent by the RFID identifier 102 of the user tag 14 and received by the RFID reader 112 of the surgical device 16 indicates that the user of the tag 14 is authorized for that surgical device 16 and/or surgical procedure and/or service.

In a service technician embodiment of tag 14, the RFID identifier 102 can include identification data and security data, as discussed above, and, in addition, calibration data, necessary to calibrate the surgical device 16. For example, calibration data may comprise instructions or parameter settings including, for example, laser calibration settings for a particular probe or adaptation. Each probe or adaptation may require the use of a different and unique optical fiber, which changes the optical coupling into the probe or adaptation and therefore results in a particular calibration. This type of information can also be included in the tag 14 embodiment for a surgeon.

As a further example, when using a vitrectomy console, calibration data may include instructions or parameter settings relating to laser probe transmissivity, vitrectomy probe pressure points and endo-illuminator transmissivity. Indeed, other calibration data may be used depending on the ophthalmic surgical device and component that are utilized. The surgical device 16 can automatically calibrate itself based on the received calibration data. Alternatively, the surgical device 16 can be calibrated by a surgeon using the received calibration data. Even further, surgical device 16 can be automatically configured (beyond calibration) and data fields pre-populated based on the data received from tag 14.

In addition, the identification and/or calibration data and/or configuration data may be used for various purposes, including for example enabling or disabling operating parameters that are compatible with the intended surgical procedure and invoking certain safety features associated with the surgical procedure or with a surgeon's desired settings. For example, a surgical system may be configured to limit power ranges and/or exposure ranges given a particular type of surgical procedure. Further, the identification and/or other data can be used to generate a user interface that is related to the desired surgical procedure, patient, surgeon, or service technician and presented on a display screen for the user.

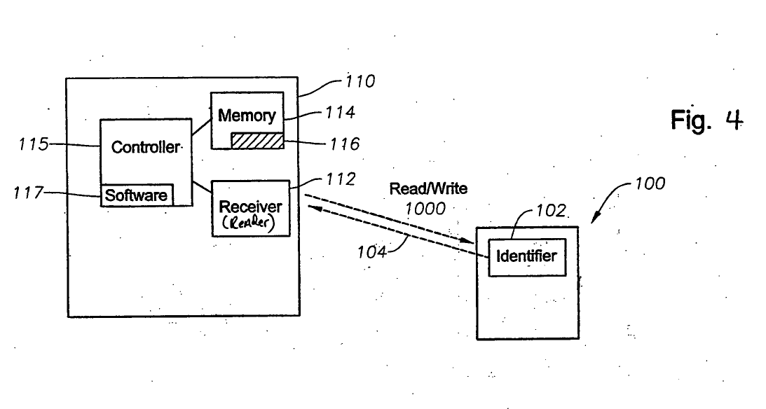

FIG. 4 is a simplified block diagram illustrating in more detail an embodiment of this invention in which the RFID readers and tags are configured for two-way read/write applications 1000. This embodiment may require greater RFID tag 102 data capabilities. In this embodiment, the reader (or transceiver) can write different types of data back to the RFID tag 102 for future use or reference. For example, the RFID reader 112 can write information, such as a date of a procedure, a patient's name, parameter settings, the elapsed procedure time, etc. This information can be particularly useful when the procedure is reviewed at a later time and/or for keeping track of maintenance schedules or for reviewing a surgical procedure for effectiveness based on the parameters used.

A person skilled in the art will appreciate that there are various modifications that can be made without departing from the scope of the invention. Embodiments can be used with other types of surgical equipment. Further, embodiments can be used for different purposes, including identification, calibration, maintenance, patient/device compatibility and lock-out purposes. Thus the illustrated examples set forth above are not intended to be limiting.

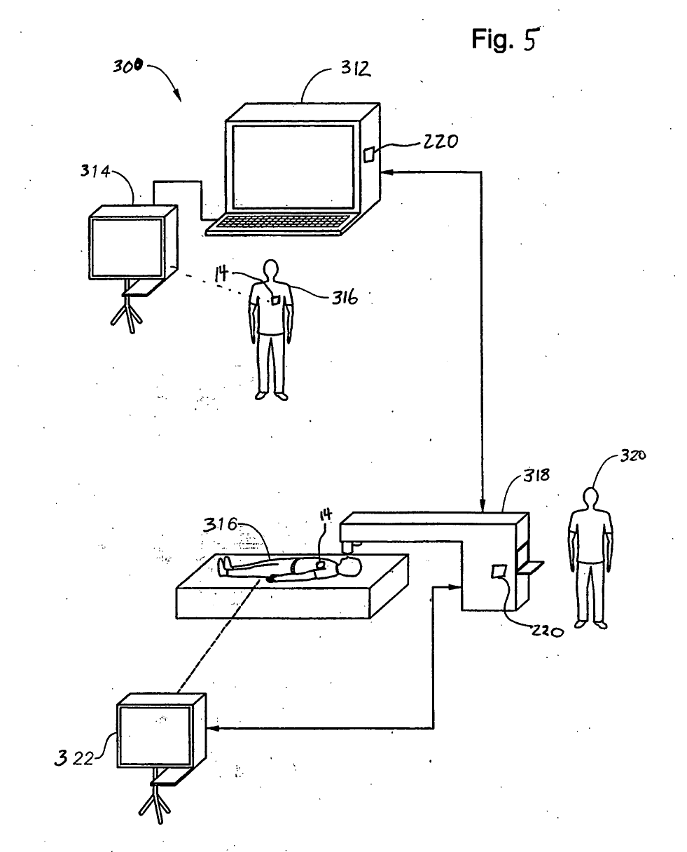

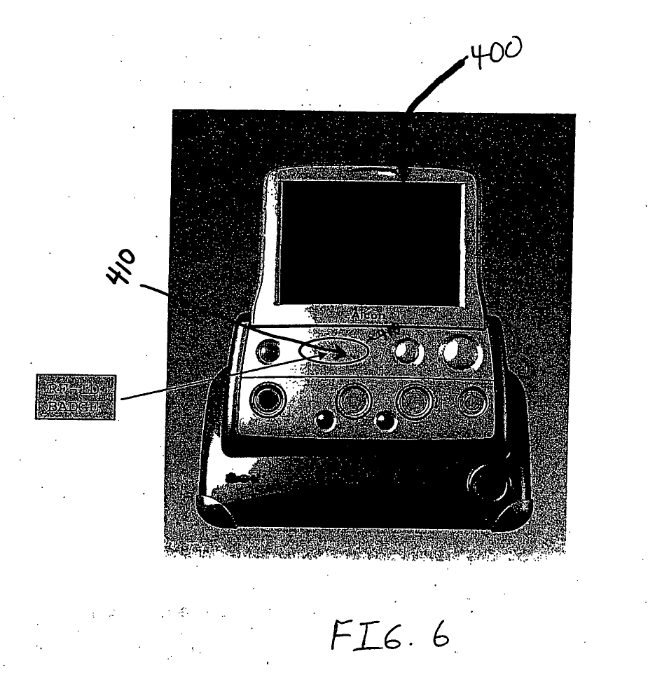

FIG. 5 is a simplified block diagram of a surgical system 300 comprising one or more input devices 314 and 322 in accordance with the teachings of this invention. Surgical system 300 further comprises a diagnostic console 312 operably coupled to input device 314. Input device 314 can be used for inputting a set of patient data associated with a patient 316. The set of data can be taken, for example, during a diagnostic procedure associated with a contemplated surgery. The diagnostic procedure can comprise, for example, a wavefront analysis for a refractive laser procedure or can be as simple as taking blood pressure, body temperature and/or any other such commonly measured patient parameters. The patient data measured and/or entered via input device 314 can comprise, for example, patient name, age, diagnosed ailment, body part to be operated on etc., or any other such data as known to those having skill in the art.

Input device 314 can provide the set of patient data to diagnostic console 312, which can associate the set of patient data with the intended surgical procedure based on, for example a doctor's entry and patient confirmation of the surgery at the time of providing the patient data. Diagnostic console 312 can comprise an input device 12 and interface 13 for loading patient information onto a tag 14, such as disclosed with reference to FIG. 1. Similarly, an input device and interface can be used to load desired surgeon data or service technician data onto a tag 14. Diagnostic console 312 can transfer the set of patient data and associated surgery data to tag 14, which is communicatively connected to diagnostic console 312 (e.g., wirelessly or via a wired interface).

Following the acquisition of this set of patient data and/or surgeon data, the set of data can be stored on tag 14 until the time at which the subsequent surgical procedure is to take place. Note that the time between a diagnostic aspect (time of entry of patient data onto a tag 14) and the surgical aspect of a surgical procedure can vary, such that the surgical aspect of the surgical procedure can immediately follow the diagnostic aspect of the surgical procedure or can occur at some later time separated by hours, days or any such time period. Once patient 316 is ready to undergo the surgical procedure, patient 316 is situated, as appropriate, on or near a surgical device 318 (analogous to surgical device 16 of FIGS. 1 and 2), depending on the surgical procedure to be performed. Before the surgery can begin, the surgeon or surgical team member 320 will either prompt patient 316 to place his associated tag 14 near the reader 220 or do it for the patient. The location of reader 220 can be indicated by, for example, a tag or other marking, or by some other indication, such as a light, indentation, touch-pad, etc., as will be known to those having skill in the art, on surgical device 318(16). Note that although described as potentially a two-step process performed at two consoles (diagnostic and surgical), the data gathering/transfer can occur at a single console in any number of steps.

Reader 220 will communicatively couple with tag 14, as described above, and transfer the respective patient/surgeon/technician data from tag 14 and provide it to the surgical device 318. Surgical device 318 is configured and data-populated based on the transferred data and can be made ready for surgery by a surgeon or other user by any other steps required for the procedure, including confirmation of the transferred data and surgical device 318 configuration. The embodiments of the method and system for configuring and data-populating a surgical device of the present invention can thus be used to effectively ensure a correct surgical procedure/patient match and an efficient surgical flow. The improvements to the efficiency and flow of a surgical procedure provided by the embodiments of this invention can be of particular value in a busy surgical practice. The embodiments of this invention can also increase safety and consistency by providing a surgeon a convenient tag 14 that includes data to reproducibly configure the surgical device 318 in accordance with the surgeon's preferences. The likelihood of a manual mistake being made from one surgery to another is thus greatly reduced or eliminated.

The embodiments of the present invention provide the advantage of streamlining the data input to and configuration of surgical equipment for a surgical procedure. Preparation for surgery can thus be made more efficient, reducing time and the likelihood of mistakes. Similar streamlining benefits and efficiency increases can be appreciated in the service and maintenance of a surgical device 318 (16). Although the proposed approach of the present invention may not replace the need for a paper record of the manually captured entries that are typically recorded by a surgeon or his assistant during a pre-exam operative procedure and post operative follow-up examinations, the embodiments of this invention will simplify the gathering of the appropriate parameters and provide a means by which there can be additional checks established to assure that a patient receives the appropriate treatment on the appropriate body part. The embodiments of this invention make it easier to capture data without having to transcribe the readings from one machine onto another or from a machine onto a patient record. The accuracy and consistency of patient records will also thus be improved.

FIG. 6 is a conceptual drawing illustrating one potential implementation of an indicator for the location of a reader 220. In this case, surgical device 400 includes an oval area 410 of the surgical device 400 casing designating where a user will place a tag 14 so that it will be in close enough proximity to communicate with reader 220. Reader 220 can be located, for example, behind the casing area 410.

The present invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims. For purposes of this description, references to a person having skill in the art mean a person of average skill in the art as intended by any relevant patent statutes.

What is claimed is:

1. A system for configuring a surgical device, comprising
an interface operable to receive data;
a portable component, comprising a radio frequency identification (RFID) tag operable to store and transmit a set of data;
a memory, the memory storing criteria; and
a controller, wherein the controller, the memory and the interface are included in the surgical device, and wherein the controller is operable to configure the surgical device based on the set of data transmitted from the RFID tag and received at the interface.

2. The system of claim 1, wherein the interface is a RFID interface comprising an RFID reader operable to transmit a radio frequency (RF) instruction to activate the RFID tag of the portable component and a microcontroller operable to cause the RFID reader to transmit the RF instruction.

3. The system of claim 1, wherein the portable component is an identification tag operable to be attached to the user.

4. The system of claim 1, wherein the criteria is user authorization data that is stored in the memory, the controller is further operable to determine whether a user associated with the portable component is authorized to use the surgical device based on whether the set of data transmitted from the REID tag and received at the interface satisfies the criteria stored in the memory, and wherein the set of data received from the RFID tag is compared to the user authorization data at the controller to determine whether the user is authorized to use the surgical device.

5. The system of claim 1, wherein the criteria is an algorithm, the controller is further operable to determine whether a user associated with the portable component is authorized to use the surgical device based on whether the set of data transmitted from the RFID tag and received at the interface satisfies the criteria stored in the memory, and wherein the controller applies the algorithm to the set of data received from the RFID tag to determine whether the user is authorized to use the surgical device.

6. The system of claim 1, wherein the RFID tag is a passive tag.

7. The system of claim 1, wherein the RFID tag is an active tag.

8. The system of claim 1, wherein the interface is operable to receive and transmit data and wherein the RFID tag is operable to receive and store a second set of data.

9. The system of claim 8, wherein the interface is operable to program the RFID tag with the second set of data, and wherein the set of data and the second set of data can be different.

10. The system of claim 1, wherein the set of data comprises data selected from the group consisting of patient identification data, surgeon custom setting data, service data, maintenance data, surgical device calibration data, and surgical device configuration data.

11. The system of claim 1, wherein configuring the surgical device comprises data-populating selected data fields based on the set of data.

12. The system of claim 1, wherein configuring the surgical device comprises setting selected surgical device parameters to predetermined values in preparation for use based on the set of data.

13. The system of claim 1, wherein configuring the surgical device comprises placing the surgical device in a service configuration in preparation for repair or maintenance.

14. The system of claim 1, further comprising an input device having an input device interface operable to program the RFID tag with the set of data and wherein the input device can be different from the surgical device.

15. The system of claim 14, wherein the interface and the input device interface are the same interface.

16. The system of claim 1, wherein the surgical device is selected from the group consisting of an ophthalmic laser, a vitreoretinal surgical device, and a phacoemulsification system.

17. The system of claim 1, wherein the controller is further operable to determine whether a user associated with the portable component is authorized to use the surgical device based on whether the set of data transmitted from the RFID tag and received at the interface satisfies the criteria stored in the memory and operable to disable the surgical device if it is determined the user is not authorized to use the surgical device.

18. The system of claim 1, wherein the set of data is encrypted and wherein the controller is operable to decrypt encrypted data.

19. A system for configuring a surgical device, comprising
an interface operable to receive data;
a portable component, comprising an identifier operable to store and transmit a set of data;
a memory, the memory storing criteria; and
a controller, wherein the controller, the memory and the interface are included in the surgical device, and wherein the controller is operable to:
determine whether a user associated with the portable component is authorized to use the surgical device based on whether the set of data transmitted from the RFID tag and received at the interface satisfies the criteria stored in the memory; and
configure the surgical device based on the set of data.

20. The system of claim 19, wherein the identifier is an RFID tag, and wherein the interface is a RFID interface comprising an RFID reader operable to transmit a radio frequency (RF) instruction to activate the RFID tag of the portable component and a microcontroller operable to cause the RFID reader to transmit the RF instruction.

22. The system of claim 19, wherein the portable component is an identification tag operable to be attached to the user.

23. The system of claim 19, wherein the criteria is user authorization data that is stored in the memory, and wherein the set of data received from the identifier is compared to the user authorization data at the controller to determine whether the user is authorized to use the surgical device.

24. The system of claim 19, wherein the criteria is an algorithm, and wherein the controller applies the algorithm to the set of data received from the identifier to determine whether the user is authorized to use the surgical device.

25. The system of claim 19, wherein the interface is a transceiver operable to receive and transmit data and wherein the identifier is operable to receive and store a second set of data.

26. The system of claim 25, wherein the interface is operable to program the identifier with the second set of data, and wherein the set of data and the second set of data can be different.

27. The system of claim 19, wherein the set of data comprises data selected from the group consisting of patient identification data, surgeon custom setting data, service data, maintenance data, surgical device calibration data, and surgical device configuration data.

28. The system of claim 19, wherein configuring the surgical device comprises data-populating selected data fields based on the set of data.

29. The system of claim 19, wherein configuring the surgical device comprises setting selected surgical device parameters to predetermined values in preparation for use based on the set of data.

30. The system of claim 19, wherein the surgical device is selected from the group consisting of an ophthalmic laser, a vitreoretinal surgical device, and a phacoemulsification system.

32. The system of claim 19, wherein the controller is further operable to disable the surgical device if it is determined the user is not authorized to use the surgical device.

33. The system of claim 19, wherein the set of data is encrypted and wherein the controller is operable to decrypt encrypted data.

34. A method for configuring a surgical device, comprising
providing a portable component, comprising an identifier operable to store and transmit a set of data;
establishing a criteria and storing the criteria in a memory of the surgical device;
transmitting the set of data stored in the identifier from the identifier to an interface operable to receive data in the surgical device;
determining at a controller in the surgical device whether a user associated with the portable component is authorized to use the surgical device based on whether the set of data transmitted from the identifier and received at the interface satisfies the criteria stored in the memory; and
configuring the surgical device based on the set of data.

35. The method of claim 34, wherein the identifier is an RFID tag, and wherein the interface is a RFID interface comprising an RFID reader operable to transmit a radio frequency (RF) instruction to activate the RFID tag of the portable component and a microcontroller operable to cause the RFID reader to transmit the RF instruction.

36. The method of claim 34, wherein the portable component is an identification tag operable to be attached to the user.

37. The method of claim 34, wherein the criteria is user authorization data that is stored in the memory, and further comprising comparing the set of data received from the identifier to the user authorization data at the controller to determine whether the user is authorized to use the surgical device.

38. The method of claim 34, wherein the criteria is an algorithm, and further comprising applying the algorithm at the controller to the set of data received from the identifier to determine whether the user is authorized to use the surgical device.

39. The method of claim 34, wherein the interface is a transceiver operable to receive and transmit data and wherein the identifier is operable to receive and store a second set of data.

40. The method of claim 39, wherein the interface is operable to program the identifier with the second set of data, and wherein the set of data and the second set of data can be different.

41. The method of claim 34, wherein the set of data comprises data selected from the group consisting of patient identification data, surgeon custom setting data, service data, maintenance data, surgical device calibration data, and surgical device configuration data.

42. The method of claim 34, wherein configuring the surgical device comprises data-populating selected data fields based on the set of data.

43. The method of claim 34, wherein configuring the surgical device comprises setting selected surgical device parameters to predetermined values in preparation for use based on the set of data.

44. The method of claim 45, programming the identifier with the set of data at an input device having an input device interface, wherein the input device can be different from the surgical device.

45. The method of claim 44, wherein the interface and the input device interface are the same interface.

46. The method of claim 34, wherein the surgical device is selected from the group consisting of an ophthalmic laser, a vitreoretinal surgical device, and a phacoemulsification system.

47. The method of claim 46, further comprising disabling the surgical device if it is determined the user is not authorized to use the surgical device.

48. The method of claim 1, wherein the set of data is encrypted and wherein the controller is operable to decrypt encrypted data.
